(19) United States
(12) Patent Application Publication
(10) Pub. No.: US 2008/0004485 A1
(43) Pub. Date: Jan. 3, 2008

(54) Trans-Septal Heart Assist Devices and Methods of Use

(76) Inventor: Rafael M. Moreschi, Cary, NC (US)

Correspondence Address: COATS & BENNETT, PLLC, 1400 Crescent Green, Suite 300, Cary, NC, 27518, US

(21) Appl. No.: 11/427,793

(22) Filed: Jun. 30, 2006

(51) Int. Cl. A61M 1/12

(52) U.S. Cl. 600/16

(57) ABSTRACT

An implant may be secured to a cardiac septum. The implant may include an anchor portion that embeds within the septum or an anchor portion with inflatable first and second anchor balloons coupled to one another through an aperture in the cardiac septum. The anchor balloons include a deflated width that is less than a width of the aperture. The anchor balloons include an inflated width that is greater than the width of the aperture. The implant includes at least one inflatable assist balloon to correct a cardiac dysfunction, such as systolic dysfunction or valve insufficiency. The assist balloon may assume a first shape during cardiac systole and a second shape during cardiac diastole. The implant includes catheter tubing including at least one lumen through which a fluid may be introduced to inflate the assist or anchor balloons. A pump and associated electronics may inflate and deflate the assist balloon.

### BACKGROUND

The human heart performs several functions, not the least of which is to collect oxygen-poor blood from the body and pump that blood to the lungs where it picks up oxygen and releases carbon dioxide. Simultaneously, the heart collects oxygen-rich blood from the lungs and pumps that blood to the body so that cells throughout the body have oxygen necessary for proper function. Various conditions may lead to insufficient function of the heart. Coronary heart disease may be the most common form of heart disease in the Western world. Other, lesser-known conditions may affect heart function with equally detrimental effects. For instance, systolic and diastolic dysfunction may represent disorders of the myocardium while aortic and mitral insufficiency may represent disorders of the heart valves. These and other types of disorders may also impair heart function.

In severe cases, cardiac transplantation or valve replacement may be indicated. Unfortunately, the wait for a donor heart delays the availability of a heart transplant. In some cases, the fragile condition of the patient may limit the availability of open-heart surgeries. Further, any type of open-heart surgery has a high morbidity and mortality, particularly in elderly patients. Open surgeries also have other associated complications, including post-surgical strokes, heart attacks, infection, and renal failure to name a few. Accordingly, a device that may be installed without open-heart surgery and that temporarily or permanently prevents and treats congestive heart failure induced by these conditions may be desirable.

### SUMMARY

Illustrative embodiments disclosed herein are directed to an implant that is insertable into a heart in a patient and includes an anchor portion. The anchor portion may include a sharpened feature that can engage or embed within a cardiac septum. Alternatively, the anchor portion may include inflatable first and second anchor balloons. The anchor balloons may be coupled to one another through an aperture in the cardiac septum. For insertion, the anchor balloons have a deflated width that is less than a width of the aperture. To secure the implant, the anchor balloons may have an inflated width that is greater than the width of the aperture. The implant may include an inflatable assist balloon to assist in cardiac function. One or both of the anchor balloons may operate as an assist balloon. The assist balloon may assume a first shape during cardiac systole and assume a second shape during cardiac diastole. The implant may further include catheter tubing including at least one lumen through which a fluid may be introduced to inflate the assist balloon or anchor balloons.

The implant may include a pumping mechanism with a fluid reservoir to contain the fluid, a pump to move the fluid between the fluid reservoir and the lumen, a sensor to sense cardiac systole and diastole rhythms, and a controller to control the operation of the pump to move the fluid in synchronization with the cardiac systole and diastole rhythms. The pumping mechanism may be subcutaneously implanted. The controller may operate the pump to inflate and deflate the assist balloon between the first and second shapes. The assist balloon may be inflated and deflated in a ventricle to assist systolic dysfunction. The assist balloon may be disposed in a left ventricle adjacent a cardiac mitral valve, with the assist balloon assuming a first shape to seal the mitral valve during systole and assuming an elongated shape during diastole to permit blood flow around the assist balloon. The assist balloon may be disposed in an aorta adjacent a cardiac aortic valve, with the assist balloon assuming a first shape to seal the aortic valve during diastole and assuming an elongated shape during systole to permit blood flow around the assist balloon.

### BRIEF DESCRIPTION OF THE DRAWINGS

FIG. 1 is a diagram of an open section of a human heart illustrating the anatomy and function of the heart;

FIG. 2 is a schematic representation of a cardiac implant according to one embodiment;

FIG. 3 is a detailed section view of anchor balloons used to secure the cardiac implant according to one embodiment;

FIG. 4 is a diagram of an open section of a human heart illustrating an installed cardiac implant according to one embodiment;

FIG. 5 is a diagram of an open section of a human heart illustrating an installed cardiac implant with anchor balloons inflated to assist in systolic flow according to one embodiment;

FIG. 6 is a diagram of an open section of a human heart illustrating an installed cardiac implant with one anchor balloon inflated to assist in systolic flow at the left ventricle according to one embodiment;

FIG. 7 is a diagram of an open section of a human heart illustrating an installed cardiac implant with one anchor balloon inflated to assist in systolic flow at the right ventricle according to one embodiment;

FIG. 8 is a diagram of an open section of a human heart illustrating an installation catheter according to one embodiment;

FIG. 9 is a diagram of an open section of a human heart illustrating a secured installation catheter and an associated cutting tool contained therein according to one embodiment;

FIG. 10 is a diagram of an open section of a human heart illustrating a secured installation catheter and an associated cutting tool forming an aperture in the intra-ventricular septum according to one embodiment;

FIG. 11 is a diagram of an open section of a human heart illustrating a secured installation catheter and a cardiac implant guided through the installation catheter towards the intra-ventricular septum according to one embodiment;

FIG. 12 is a diagram of an open section of a human heart illustrating an installed cardiac implant with an assist balloon assuming a shape to seal the aortic valve according to one embodiment;

FIG. 13 is a diagram of an open section of a human heart illustrating an installed cardiac implant with an assist balloon assuming a shape to allow aortic blood flow according to one embodiment;

FIG. 14 is a detailed section view of an assist balloons used to seal a cardiac valve according to one embodiment;

FIG. 15 is a detailed section view of an assist balloons used to seal a cardiac valve according to one embodiment;

FIG. 16 is a diagram of an open section of a human heart illustrating an installed cardiac implant with an assist balloon assuming a shape to seal the aortic valve according to one embodiment;

FIG. 17 is a diagram of an open section of a human heart illustrating an installed cardiac implant with an assist balloon assuming a shape to allow aortic blood flow according to one embodiment;

FIG. 18 is a diagram of an open section of a human heart illustrating an installed cardiac implant with an assist balloon assuming a shape to seal the aortic valve according to one embodiment;

FIG. 19 is a diagram of an open section of a human heart illustrating an installed cardiac implant with an assist balloon assuming a shape to allow aortic blood flow according to one embodiment;

FIG. 20 is a diagram of an open section of a human heart illustrating an installed cardiac implant with an assist balloon assuming a shape to allow blood flow through the mitral valve according to one embodiment;

FIG. 21 is a diagram of an open section of a human heart illustrating an installed cardiac implant with an assist balloon assuming a shape to seal the mitral valve according to one embodiment;

FIG. 22 is a diagram of an open section of a human heart illustrating an installed cardiac implant with an assist balloon assuming a shape to seal the mitral valve according to one embodiment; and

FIG. 23 is a diagram of an open section of a human heart illustrating an installed cardiac implant with an assist balloon assuming a shape to allow aortic blood flow according to one embodiment.

### DETAILED DESCRIPTION

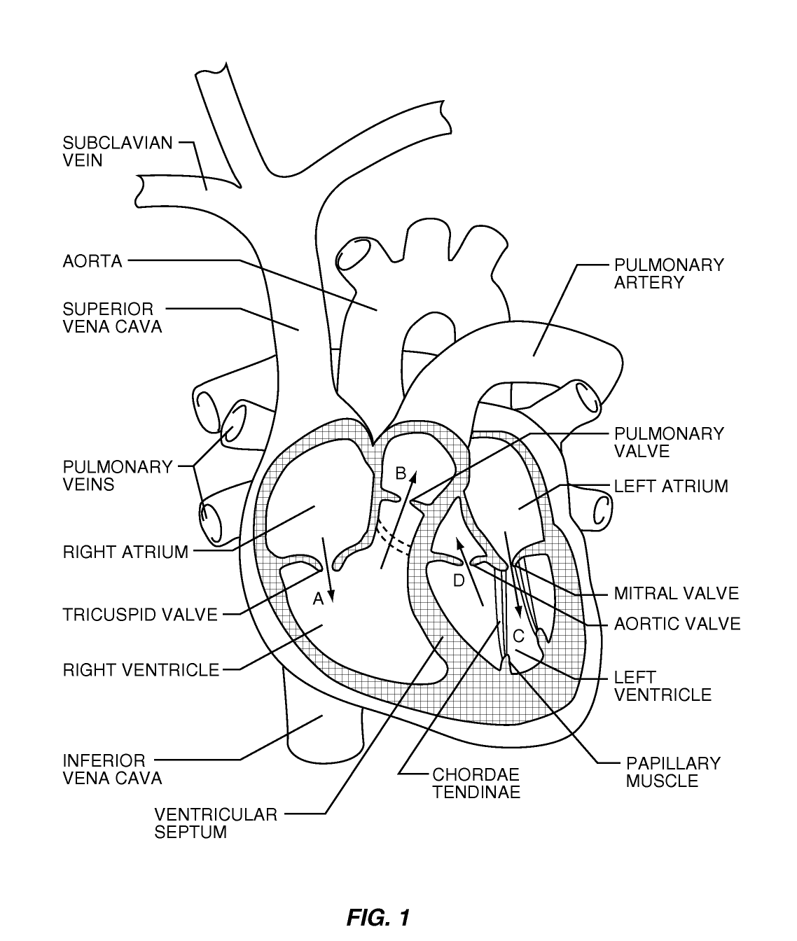

The various embodiments disclosed herein relate to a device that may be implanted into a heart such as that shown in FIG. 1. For purposes of describing the operation of the various devices, FIG. 1 generally identifies the anatomy of the heart and surrounding vascular system. FIG. 1 also shows the direction of blood flow as indicated by the arrows labeled A, B, C, and D. Briefly, the heart is divided into 4 chambers: Right Atrium, Right Ventricle, Left Atrium, and Left Ventricle. Each chamber includes a valve at its exit that prevents blood from flowing backwards. When each chamber contracts the valve at its exit opens. When the chamber is finished contracting, the valve closes so that blood does not flow backwards. The valves include the Tricuspid valve at the exit of the Right Atrium, the Pulmonary valve at the exit of the Right Ventricle, the Mitral valve at the exit of the Left atrium, and the Aortic valve at the exit of the Left Ventricle. FIG. 1 further depicts Papillary Muscles coupled to the Mitral Valve via Chordae Tendinae to assist Mitral Valve function.

When the heart muscle contracts (called systole), it pumps blood out of the heart. The heart contracts in two stages. In the first stage the Right and Left Atria contract at the same time, pumping blood to the Right and Left Ventricles, respectively. Then, the Ventricles contract together to propel blood out of the heart. Then the heart muscle relaxes (called diastole) before the next heartbeat. This allows blood to fill the heart again. Oxygen-poor blood enters the Right Atrium from the Superior Vena Cava and the Inferior Vena Cava. When the Right Atrium contracts, the blood goes through the Tricuspid Valve and into the Right Ventricle. When the Right Ventricle contracts, blood is pumped through the Pulmonary Valve, into the Pulmonary Artery, and into the lungs where it picks up oxygen.

Blood returns to the heart from the lungs by way of the Pulmonary Veins and goes into the Left Atrium. When the Left Atrium contracts, blood travels through the Mitral Valve and into the Left Ventricle. The Left Ventricle is a very important chamber that pumps blood through the Aortic Valve and into the Aorta, which receives all the blood that the heart has pumped out and distributes it to the rest of the body. The Left Ventricle generally includes a thicker muscle than any other heart chamber because it must pump blood to the rest of the body against much higher pressure.

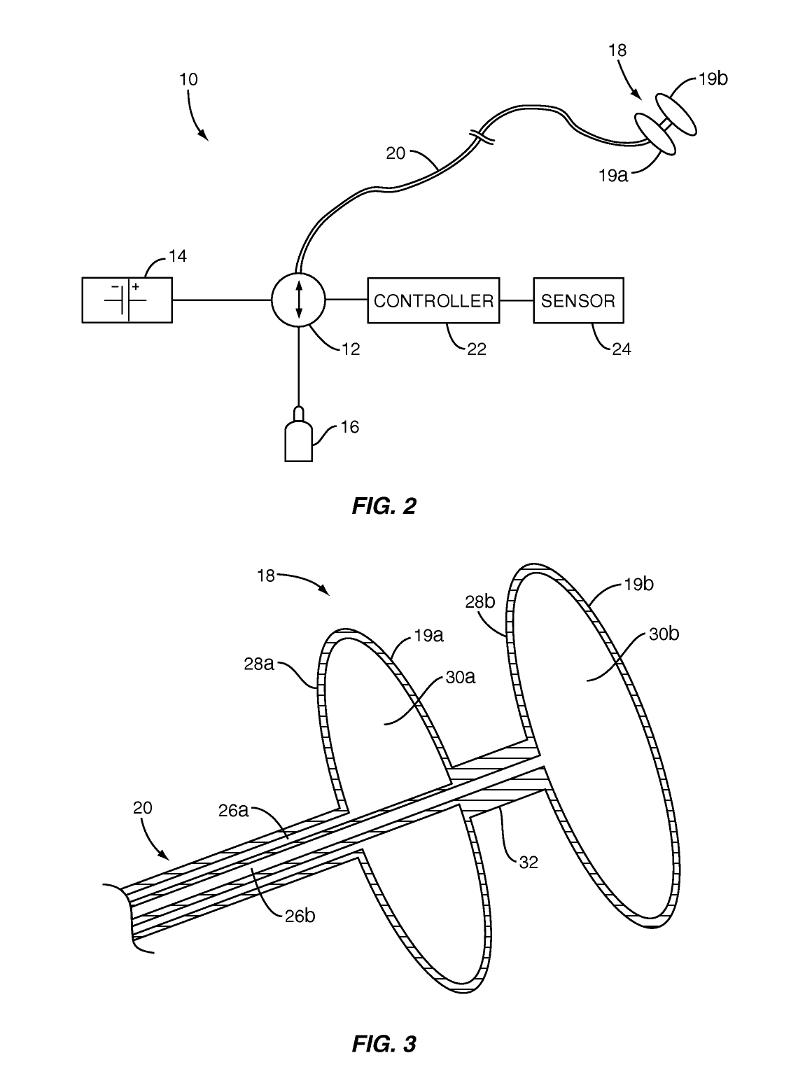

Systolic dysfunction generally refers to a condition resulting from decreased contractility of the cardiac muscle causing the ventricles to lose the ability to eject blood. In many instances, systolic dysfunction affects the left ventricle and its ability to eject blood into the high pressure aorta. However, in other patients, systolic dysfunction may also affect the right ventricle and the ability to eject blood into the pulmonary arteries. FIGS. 2 and 3 depict an implant device 10 that may be used to assist systolic pumping for the left ventricle, the right ventricle, or both.

The device 10 generally includes a pump 12 that is powered by an energy source 14 and is configured to reversibly pump a biocompatible fluid between a reservoir 16 and inflatable balloon portion 18. In one embodiment, the biocompatible fluid is contained within a closed system formed between the balloon portion 18 and the reservoir 16. In various embodiments disclosed herein, the balloon portion 18 is implanted in the heart and the individual balloons 19a, 19b are inflated and deflated to assist various cardiac functions. In one embodiment, the device 10 is implanted to assist with systolic dysfunction and the balloons 19a, 19b are positioned into the left and right ventricles. In other embodiments described herein, the balloon portion 18 is disposed at a heart valve to assist with valve dysfunction, such as regurgitation or insufficiency.

The biocompatible fluid flows through a tubular structure 20 between the pump 12 and the balloons 19a, 19b. The tubular structure 20 is thin and generally flexible. As such, the tubular structure 20 may pass intravenously from the pump 12 to the balloon portion 18. A controller 22 manages the operation of the pump 12 and works in conjunction with one or more sensors 24 to inflate and deflate the balloon portion 18 in synchronization with the hearts normal rhythm.

In one embodiment, the pump 12 is an electrical pump and is operated under the control of controller 22 with power provided by batteries 14. The pump 12, reservoir 16, controller 22 and batteries 14 may be implanted subcutaneously, on the anterior chest wall close to the pectorialis muscle. This proximity to the surface of the skin may permit recharging of the batteries 14 or repair of the system. The components may be implanted in other internal locations, including for example, the abdomen. The sensor 24 may be positioned about the exterior of the heart or in other locations where electrocardio signals may be sensed. For temporary implementations, the pump 12, reservoir 16, controller 22, or batteries 14 may be located external to the patient.

The biocompatible fluid may be a liquid or a gas with each providing different advantages over the other. For instance, the compressibility of a liquid such as saline may be different than the compressibility of a gas, such as carbon dioxide. Thus, for a given amount of pump 12 pressure, a gas may expand more than a liquid. Different implementations may use gases alone, liquids alone, or one in combination with the other.

In one specific embodiment, the tubing 20 and balloon portion 18 may form a contiguous volume such that as the pump 12 forces the biocompatible fluid from the reservoir 16, through the tubing 20, and into the balloon portion 18, each individual balloon 19a, 19b inflates and deflates under the influence of a common fluid pressure. The individual balloons 19a, 19b may have substantially similar structures (e.g., size and wall thickness) so that they inflate to a similar size under the same fluid pressure. In other embodiments, the individual balloons 19a, 19b may have a different size or different wall thickness so that they inflate to different sizes under the influence of the same or similar fluid pressure.

In another embodiment, the balloons 19a, 19b may be separate from one another. The pump 12 may include separate channels that are separately controllable by controller 22 to inflate and deflate the balloons 19a, 19b independent of one another. FIG. 3 depicts a balloon portion 18 according to one embodiment in which separate balloons 19a, 19b are filled via different lumens 26a, 26b in the tubing 20. A stem portion 32 interconnects the balloons 19a, 19b. The stem portion 32 may be flexible, but inexpandable or minimally expandable to seal the aperture in the inter-ventricular septum. The balloons 19a, 19b include a flexible, expandable outer wall 28a, 28b. The outer surface may or may not assume any particular shape when unfilled. The balloons 19a, 19b may be constructed in a variety of ways, including techniques utilized in other conventionally known biomedical applications, such as balloon angioplasty. The balloons 19a, 19b comprise a suitable complaint biocompatible material, such as a polymer that may include nylon, polyethylene, polyurethane, silicone, polyethylene, polypropylene, polyimide, and polyamide. The balloon portion 18 and tubing 20 may be reinforced with concentric layers of similar or dissimilar materials and/or fabrics. Generally, the fluid capacity and amount by which the balloons 19a, 19b are inflated will be case specific. However, these amounts are determinable and exemplary figures are disclosed in U.S. Pat. No. 4,861,330, the contents of which are incorporated by reference herein. For example, an assist balloon used to improve systolic function may be filled to a pressure of approximately 160 mm Hg or some value greater than systolic pressure in the patient. The assist balloon may be inflated to approximately 10-20 cc, though smaller or larger volumes may be appropriate depending on the application. The tubing 20 may include a configuration found in commonly available catheter devices.

In the embodiment shown in FIG. 3, lumen 26a is in fluid communication with interior cavity 30a in balloon 19a. Similarly, lumen 26b is in fluid communication with interior cavity 30b in balloon 19b. The lumens 26a, 26b may be disposed in a concentric manner as shown or in a parallel configuration within tubing 20. Furthermore, while two balloons 19a, 19b and two fluid lumens 26a, 26b are shown in FIG. 3, other implementations may include one lumen or three or more lumens and one balloon or three or more balloons. Further, each lumen may correspond to one or more balloons depending on a particular implementation.

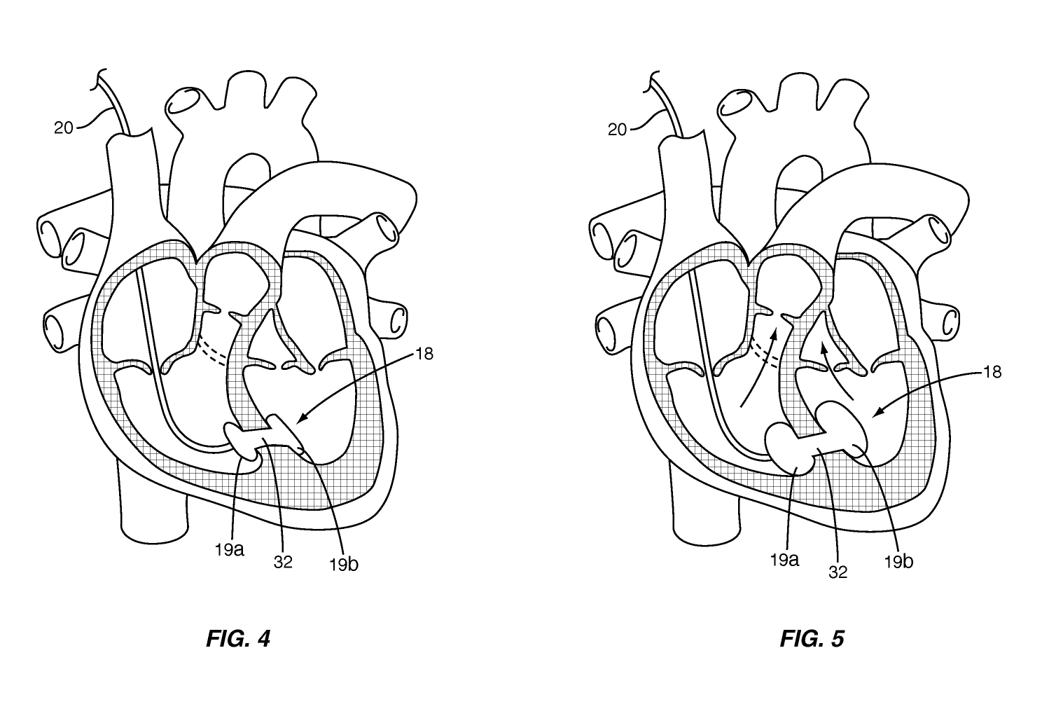

FIG. 4 shows an exposed view of the heart similar to FIG. 1 with the exemplary device 10 implanted therein. For clarity, only the tubing 20 and balloon portion 18 are shown. As shown, the balloons 19a, 19b are implanted into the right ventricle and left ventricle, respectively, with the stem portion 32 bridging an aperture that is formed in the inter-ventricular septum. The tubing 20 passes intravenously into the heart via the superior vena cava. In other implementations, the tubing may 20 may pass into the heart via other chambers, veins, or arteries, including the aorta. However, in the embodiment shown, the tubing 20 may be introduced from the subclavian vein (not shown) as an advantageous access point. A proximal end of the tubing 20 may be anchored to a rib to further secure the tubing 20. The tubing 20 passes through the tricuspid valve between the right atrium and the right ventricle. The leaflets of the tricuspid valve should be able to conform around the tubing 20 as the valve closes to minimize regurgitant effects.

In FIG. 4, both balloons 19a, 19b are shown in a partially deflated state. As described below, the balloons 19a, 19b may be completely deflated during installation and at least partially inflated to anchor the balloon portion 18 to the inter-ventricular septum as shown. In one implementation discussed above, both balloons may be inflated and deflated to assist ventricular pumping. Accordingly, FIG. 5 illustrates one configuration where both balloons 19a, 19b are inflated as compared to the configuration shown in FIG. 4.

The controller 22 from FIG. 2 operates the pump 12 in conjunction with sensed activity from sensor 24 to inflate and deflate the balloons 19a, 19b in synchronization with the QRS complex. More specifically, the balloons 19a, 19b are filled with fluid from reservoir 16 to expand at times corresponding to ventricular contraction (systole). With each heartbeat, the balloons 19a, 19b occupy increased volume within the left and right ventricles. Consequently, the instantaneous pressure within each ventricle increases in synchronization with normal systole to force blood out of each ventricle. Thus, the right atrium forces an increased amount of blood toward the pulmonary arteries and the left atrium forces an increased amount of blood toward the aorta, thereby increasing cardiac output.

During diastole, when the ventricles relax, the pump 12 reverses the direction of fluid flow to deflate the balloons 19a, 19b back to the state shown in FIG. 4. Rapid deflation may be desirable so as not to inhibit the inflow of blood. Thus, negative pump 12 pressure may be appropriate to draw a vacuum and deflate the balloons 19a, 19b. However, it may be possible to implement a pressure release valve (not shown) that is actuated in diastole to cause the balloons 19a, 19b to deflate.

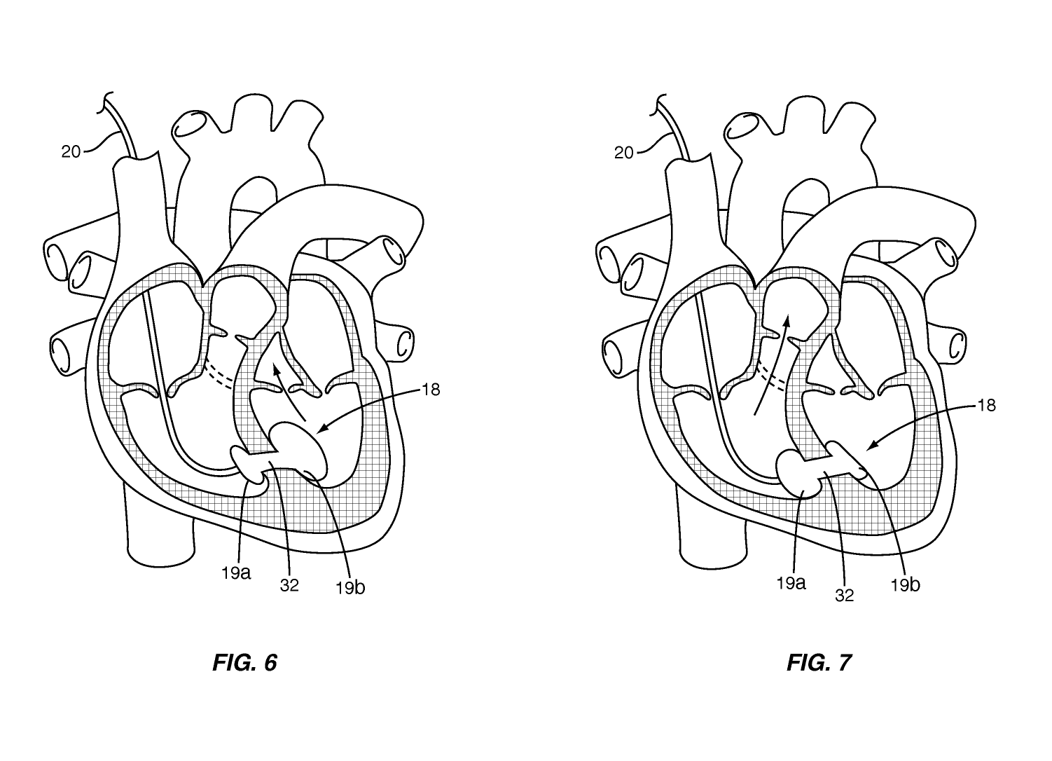

As suggested above, the individual balloons 19a, 19b may be formed so they are in fluid communication with each other. As such, they will inflate and deflate in unison between the states shown in FIGS. 4 and 5. FIG. 3 depicts an embodiment where the balloons 19a, 19b are filled by different fluid lumens 26a, 26b. Accordingly, each may be inflated or deflated independent of the other. For example, FIG. 6 shows one embodiment in which balloon 19b is inflated and deflated in conjunction with the QRS complex as described above to assist in left ventricular output. By comparison, balloon 19a is filled to a partially inflated state either during implantation or by pump 12 and retained in that state. That is, the pump 12 may maintain fluid pressure within balloon 19a to maintain the partially inflated state. Conversely, the balloon 19a may be filled independent of the pump 12, such as during surgical implantation and permanently sealed. In any event, the balloon 19a, by way of its partially inflated state, anchors the balloon portion 18 to the inter-ventricular septum. In another embodiment shown in FIG. 7, balloon 19b is filled to the partially inflated state and retained in that state while balloon 19a is inflated and deflated as described above to assist in right ventricular output.

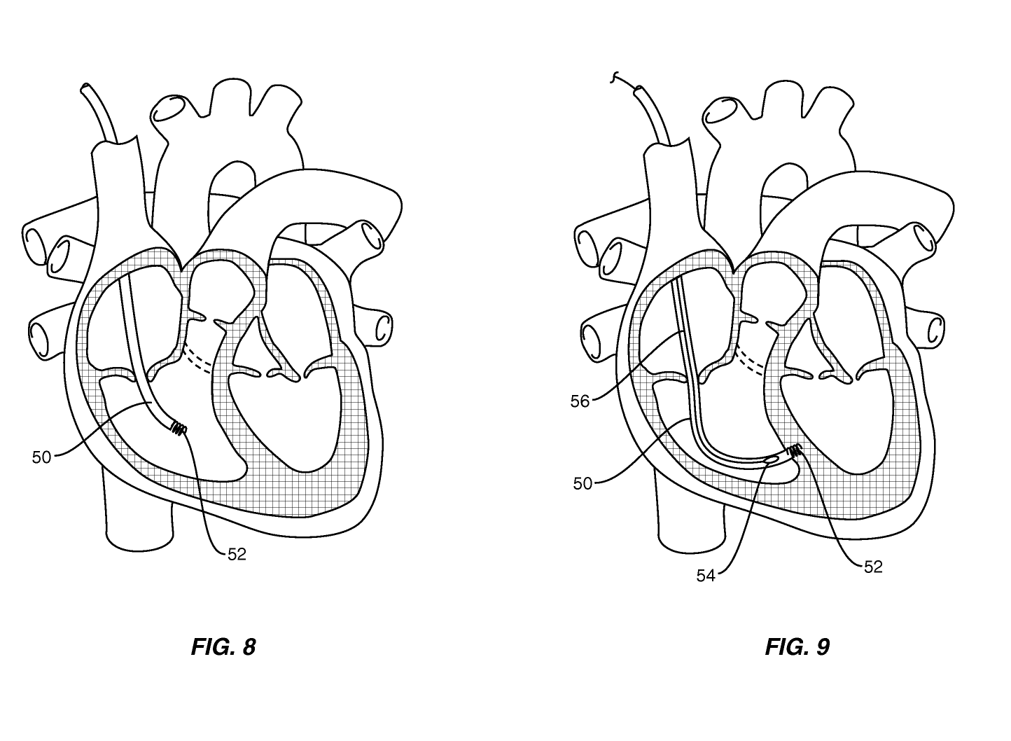

A number of techniques may be used pierce the inter-ventricular septum. For instance, a cutting tool may be guided through a catheter lumen to pierce the inter-ventricular septum. The cutting tool may be a cauterizing device including an agent or instrument to destroy tissue by burning, searing, or scarring, including caustic substances, electric currents, lasers, and very hot or very cold instruments to form an aperture in the inter-ventricular septum. FIGS. 8-11 illustrate one method by which the inter-ventricular septum may be prepared to receive the balloon portion 18 of device 10. In this particular approach, a catheter 50 is directed through the superior vena cava, through the right atrium, past the tricuspid valve, and into the right ventricle. The catheter 50 includes an anchor member 52 disposed at its distal end. The anchor member 52 is configured to at least temporarily secure the catheter 50 to the inter-ventricular septum during the implantation process. In the illustrated embodiment, the anchor member 52 is formed in a shape similar to a corkscrew, though other configurations are possible. For instance, the anchor member 52 may include a plurality of teeth or spikes or other sharpened feature capable of piercing or penetrating the septum. The anchor member 52 is guided with the aid of video fluoroscopy or other known imaging technique towards the right ventricular side of the inter-ventricular septum. Alternatively, the catheter 50 and anchor member 52 may be guided through the aorta and into the left ventricle towards the left ventricular side of the inter-ventricular septum. The catheter 50 and the corkscrew-shaped anchor member 52 are rotated to embed the anchor member 52 into the inter-ventricular septum as shown in FIG. 9.

It is contemplated that the catheter 50 is hollow in construction and sized to allow a deflated balloon portion 18 of the device 10 to pass therein. Further, as FIG. 9 shows, the catheter 50 is sized to allow a cutting member 54 to pass therein. The cutting member 54 is attached to the distal end of a guide wire 56. Using the guide wire 56, the cutting member 54 may be advanced within the catheter 50 towards the inter-ventricular septum. As indicated above, the cutting device may include a sharped blade to cut an aperture through the inter-ventricular septum. Alternatively, other cutting mechanisms may be employed, such as by burning, searing, or scarring, including caustic substances, electric currents, lasers, and very hot or very cold instruments. In any event, the cutting member 54 is advanced through the inter-ventricular septum. In some implementations, the cutting member 54 is advanced by pushing and/or rotating the guide wire. Ultimately, the cutting member 54 protrudes through the inter-ventricular septum and into the left ventricle. Once the aperture P is formed through the inter-ventricular septum, the cutting member 54 is removed through the catheter 50.

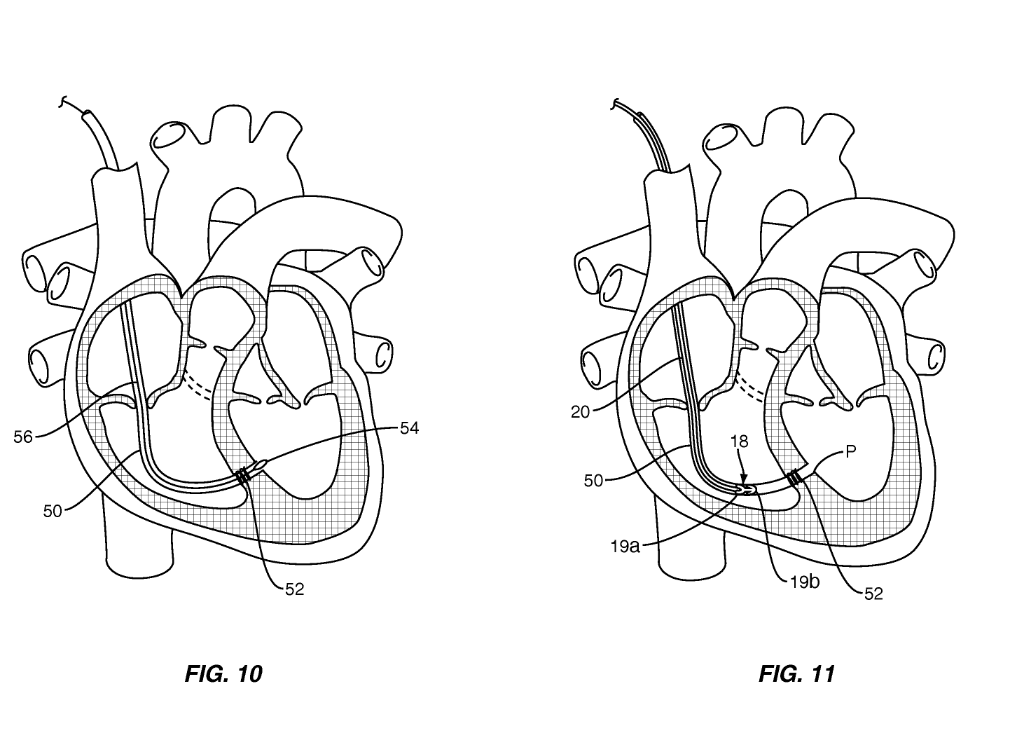

FIGS. 10 and 11 illustrate that the aperture P formed by the cutting member 54 is smaller in width than the anchor member 52. Consequently, the anchor member 52 remains seated within the inter-ventricular septum after the aperture P is formed. Once the cutting member 54 and guide wire 56 are removed, the tubing 20 and balloon portion 18 of the device 10 may be inserted through the larger diameter catheter 50 and positioned across the inter-ventricular septum. The balloon portion 18 may be completely deflated to pass through the catheter 50. The deflated balloon portion 18 includes a deflated width that is smaller than the width of aperture P. However, once the balloon portion 18 is positioned as desired, the outermost (i.e., most distal balloon 19b) may be partially inflated so that the balloon 19b assumes an inflated width that is greater than the width of aperture P. Then, the catheter 50 and anchor member 52 may be removed. In the embodiment shown, the catheter 50 is removed by unscrewing the cork-screw style anchor member 52. Since the most-distal balloon 19b is partially inflated, the balloon portion 18 remains anchored to the inter-ventricular septum and resists being pulled out along with the catheter 50. Once the catheter 50 and anchor member 52 are removed, the more-proximal balloon 19a may be partially inflated as well to completely anchor the balloon portion 18 to the inter-ventricular septum as shown in FIG. 4.

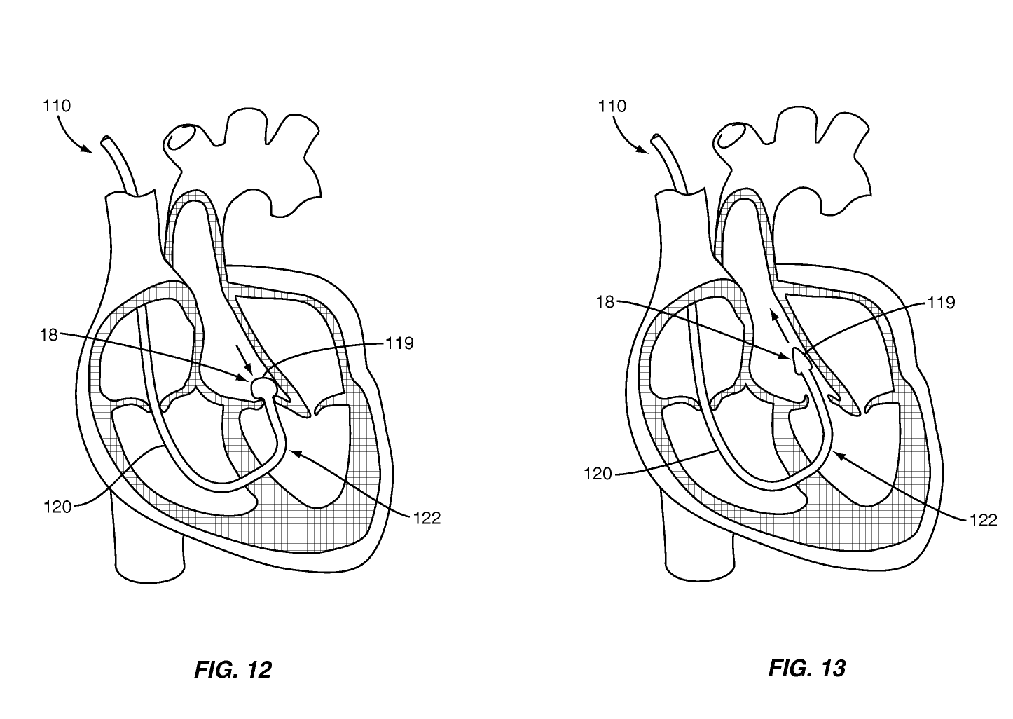

FIGS. 12 and 13 illustrate one embodiment of a cardiac assist device 110 that is used to minimize aortic regurgitation (or aortic insufficiency). FIGS. 12 and 13 illustrate a slightly different cross section of the exemplary heart taken behind the pulmonary artery to more clearly depict the aorta and aortic valve. Aortic regurgitation refers to a condition in which the cusps of the aortic valve do not seal properly. As a result, when the heart relaxes in diastole, the aortic valve does not close properly, allowing blood to regurgitate into the left ventricle. To compensate for this condition, the device 110 comprises a balloon 119 that is disposed at a distal end of a catheter tube 120. The structure of the device 110 is similar to the previously described embodiments in that the tubing 120 is hollow to allow a biocompatible fluid to flow between a pump 12 and the balloon 119. The balloon 119 may be inflated and deflated in synchronization with the systole and diastole rhythms as described before. However, in contrast with the previous embodiments, the balloon 119 is inflated in diastole to assist in sealing the aortic valve as shown in FIG. 12. Similarly, the balloon 119 is at least partially deflated in systole to reduce the size of the balloon 119 and permit additional blood flow around the balloon 119 as shown in FIG. 13. The pressures and/or volumes to which the balloon 119 is inflated will vary by application. However, it may be appropriate for the balloon to occupy between about 2-6 square centimeters of the cross section of an aorta. Appropriate pressures may be between about 5-25 mm Hg, though larger or greater numbers are possible.

FIG. 12 specifically shows that the balloon 119 is disposed at the distal end of the tubing 120. As with the previously described embodiments, the tubing 120 is inserted through the superior vena cava, through the right atrium, through the right ventricle, across the inter-ventricular septum, through the left ventricle, through the aortic valve, and into the aorta. The device 110 may be inserted from different approaches, including from the aorta. In either case, a left ventricular portion 122 of the tubing 120 passes through the left ventricle between the inter-ventricular septum and the balloon 119. The length of this portion 122 of the tubing allows the balloon 119 to move between a first position where it is in contact with the aortic valve as in FIG. 12 and a second position where the balloon 119 is downstream of the aortic valve as in FIG. 13. In one embodiment, this portion 122 may be about four to six inches in length, though the length required for a particular implementation may be different.

FIG. 12 shows the heart in diastole, where the balloon 119 is inflated and the aortic valve is closed. It is contemplated that the balloon 119 is inflated by an amount that closes the regurgitant orifice yet is compliant enough to conform to the anatomy of the leaflets in the aortic valve during diastole. By comparison, FIG. 13 shows the heart in systole, where the balloon 119 is displaced into the aorta and away from the aortic valve, which is open. When deflated, the balloon 119 may take a fusiform shape that permits blood to flow around the balloon 119.

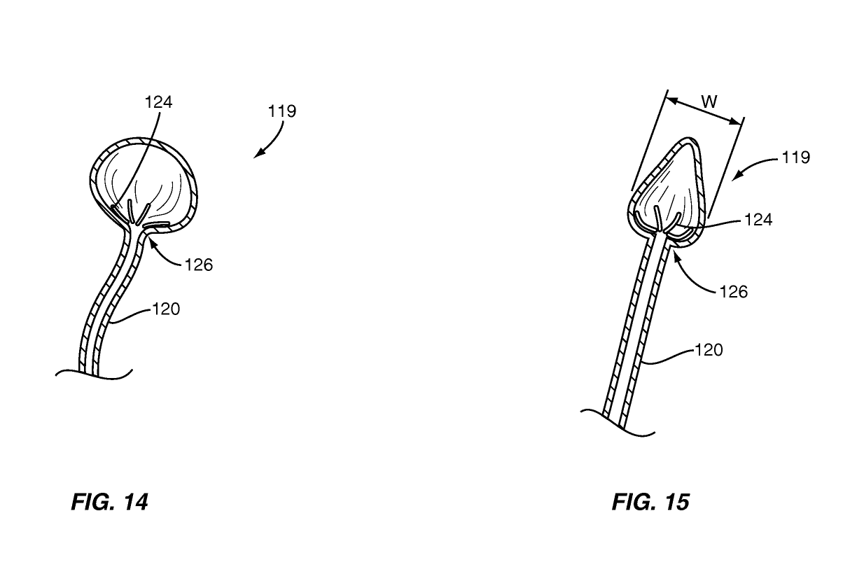

FIGS. 14 and 15 provide a cross section view of one embodiment of the balloon 119. Specifically, FIG. 14 shows the balloon 119 in an inflated state while FIG. 15 shows the balloon 119 in a partially deflated state. The balloon 119 is formed from a compliant material as described above. However, the balloon 119 may include a reinforcement portion 124 that limits the width W to which the balloon 119 deflates during systole. The limited deflation width W may serve to prevent the balloon 119 from becoming displaced back into the left ventricle, particularly with large aortic insufficiency gradients and aortic valve openings. In one embodiment, the reinforcement portion 124 is comprised of a plurality of laterally extending ribs disposed inside, outside, or within the balloon 119 material that limit the deflation width W of the balloon 119. In other embodiments, the transition region 126 between the tubing 120 and the balloon 119 may be reinforced with a more rigid section of material that at least partially retains its shape as the balloon 119 is inflated and deflated.

The balloon 119 may be inflated and deflated using a biocompatible fluid as described above. For instance, a gas such as carbon dioxide, air, or helium may provide rapid inflation and deflation times. However, in another embodiment, the balloon 119 may be partially inflated to a predetermined pressure and volume with a more dense fluid such as saline and retained at that pressure and volume. In other words, in an alternative implementation, the balloon 119 is not inflated and deflated during diastole and systole, respectively, as described above. Instead, the balloon 119 is partially inflated to a deformable condition so that the balloon 119 will take a fusiform shape in systole and flatten against the aortic valve in diastole when the aortic valve closes. FIGS. 12 and 13 appropriately depict this implementation in addition to the cyclically inflated balloon 119 embodiment. Furthermore, the reinforcement portion 124 depicted in FIGS. 14 and 15 may be used in this latter embodiment as well. Note that where cyclic inflation and deflation is not used, the desired pressure and volume may be set during implantation and the device 110 may be implanted without the pump 12, reservoir 16 and associated electronics.

Installation of the device 110 may be similar to that described above for device 10. In one implantation procedure, the aperture P in the inter-ventricular septum may be formed as shown in FIGS. 8-10. Further, the balloon 119 may be introduced into the heart as shown in FIG. 11. However, for devices 110 used to treat aortic regurgitation, the balloon 119 is advanced completely into the left ventricle. Next, a small amount of gas may be injected into the balloon 119 to at least partially inflate the balloon 119. A gas such as carbon dioxide, air, or helium may be used. The gas inside the balloon 119 may cause the balloon 119 to float on top of the blood in the left ventricle. Since the balloon 119 is only partially inflated, the balloon 119 may pass through the aortic valve due to the pressure gradient that exists during systole.

Next, in one embodiment, the balloon 119 may be inflated a slightly greater amount to prevent the balloon 119 from passing back through the aortic valve and into the left ventricle. Then, the balloon 119 may be inflated and deflated in synchronization with the systole and diastole rhythms of the heart. In another embodiment, the balloon 119 may be emptied of all gas and partially filled with a liquid such as water or saline and retained at a desired pressure and volume.

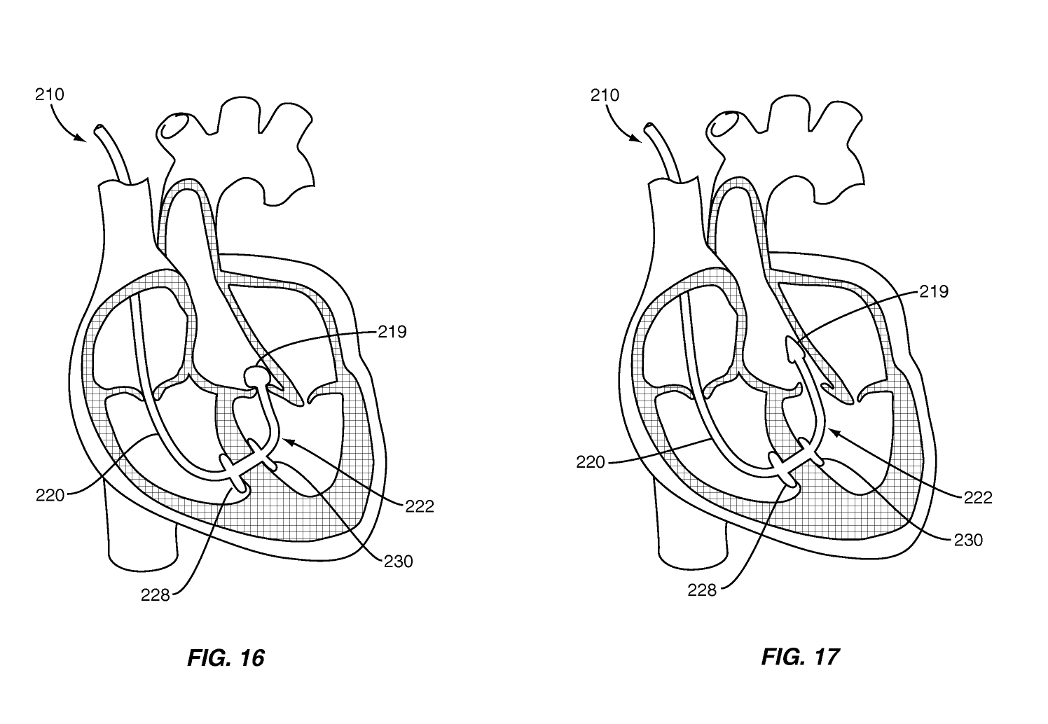

FIGS. 16 and 17 illustrate one embodiment of a device 210 that is used to treat aortic regurgitation. The device 210 is similar to device 110 depicted in FIGS. 12 and 13 except that anchors are incorporated in device 210 to secure the device 210 to the heart. Specifically, anchor balloons 228, 230 are incorporated to secure the tubing 220, including the left ventricular portion 222 of the tubing 220 to the inter-ventricular septum. The anchor balloons 228, 230 may be filled with a sufficient amount of fluid to conform to and remain in supported contact with the walls of the inter-ventricular septum. With the anchor balloons 228, 230 in relatively fixed positions, the left ventricular portion 222 of the tubing 220 should have a sufficient amount of slack to permit movement of the balloon 219 between the two positions indicated in FIGS. 16 and 17.

Notably, FIG. 16 shows the heart in diastole, where the balloon 219 is positioned adjacent the aortic valve to prevent regurgitation. As with the embodiment shown in FIGS. 12 and 13, the balloon 219 may be maintained at a predetermined pressure and volume or inflated and deflated in synchronization with the natural systole and diastole rhythms of the heart. For either case, it is contemplated that the balloon 119 is inflated by an amount that closes the regurgitant orifice yet is compliant enough to conform to the anatomy of the leaflets in the aortic valve during diastole. By comparison, FIG. 17 shows the heart in systole, where the balloon 219 is displaced into the aorta and the aortic valve is open. During systole, the pressure gradient may cause the balloon 219 to take a fusiform shape that permits blood to flow around the balloon 219. Installation of the device 210 may be substantially similar as that described above for device 110. However, the anchor balloons 228, 230 may be filled with a liquid such as water or saline and retained at a desired pressure and volume to anchor the tubing 220. In one embodiment, the balloon 219, and anchor balloons 228, 230 are in fluid communication with one another. In another embodiment, the balloon 219 may be inflated via a dedicated lumen separate from that used to fill the anchor balloons 228, 230.

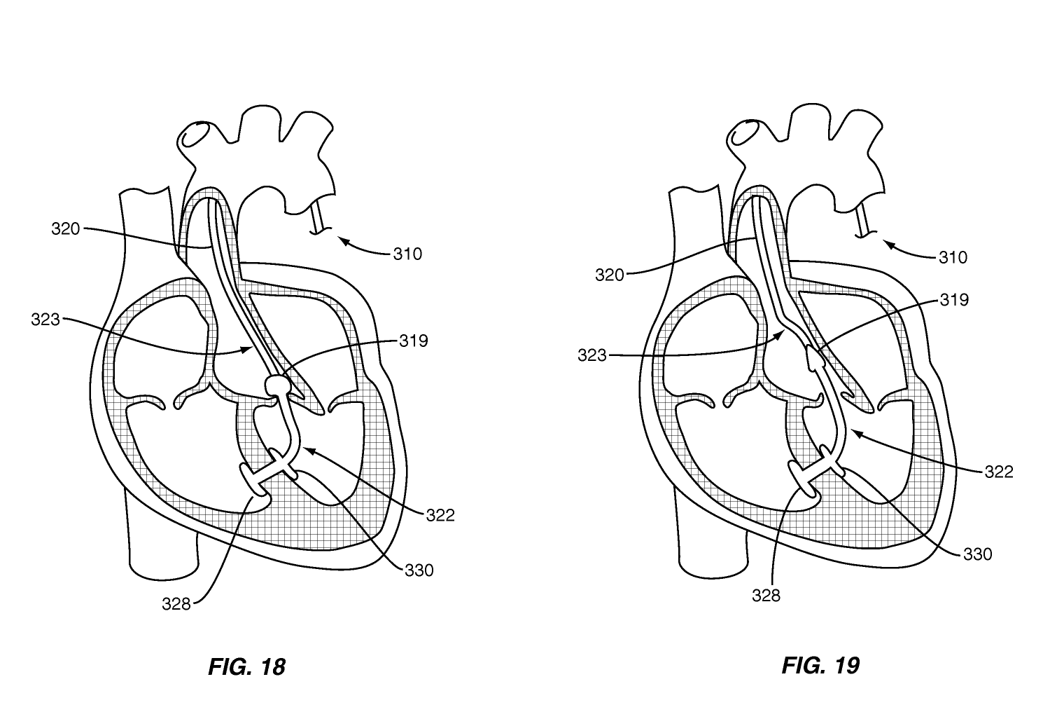

Embodiments disclosed above have generally incorporated a catheterized device that is introduced to the heart from the right atrium. However, as mentioned, the balloon devices may be introduced via the aorta as depicted in the embodiment shown in FIGS. 18 and 19. Device 310 is used to treat aortic regurgitation and includes tubing 320 that is introduced into the heart through the aorta. The distal end of the device 310 includes two anchor balloons 328, 330 that are used to secure the device to the inter-ventricular septum. A left ventricular portion 322 of the tubing 320 is proximal the anchor balloons 328, 330 and passes through the left ventricle and through the aorta to a balloon 319 that functions similar to balloon 119, 219 described above. Notably, FIG. 18 shows the heart in diastole, where the balloon 319 is positioned adjacent the aortic valve to prevent regurgitation. FIG. 19 shows the heart in systole, where the balloon 219 is displaced into the aorta and the aortic valve is open. Thus, the left ventricular portion 322 of the tubing 320 and an aortic portion 323 of the tubing 320 should have a sufficient amount of slack to permit movement of the balloon 319 between the two positions indicated in FIGS. 18 and 19.

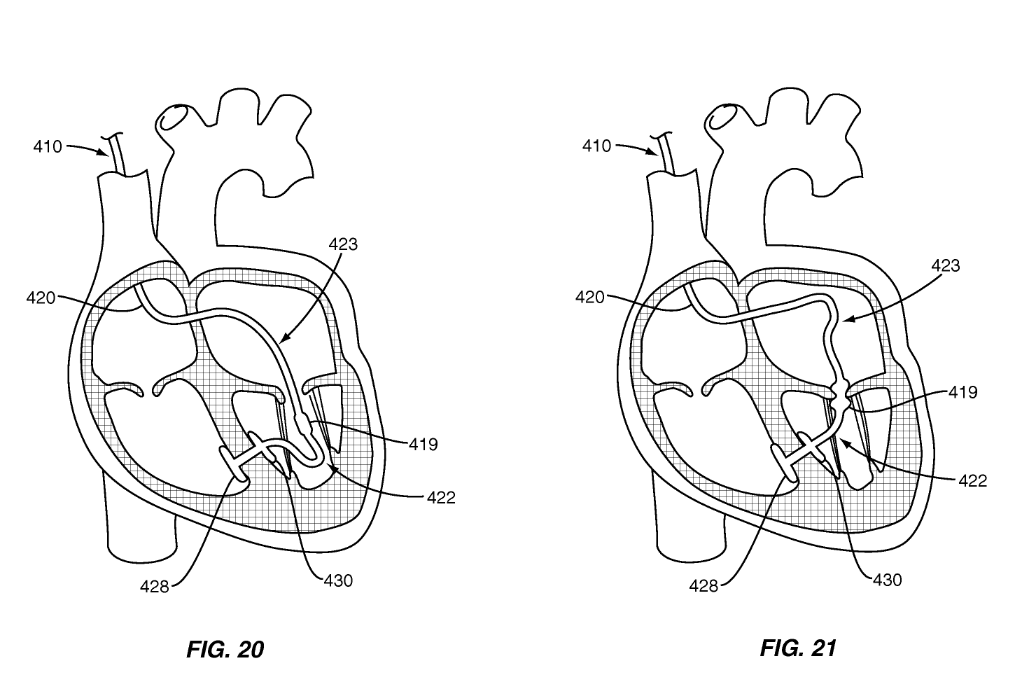

FIGS. 20 and 21 illustrate one embodiment of a cardiac assist device 410 that is used to minimize mitral regurgitation (or mitral insufficiency). FIGS. 20 and 21 illustrate a slightly different cross section of the exemplary heart taken behind the aorta to more clearly depict the left and right atria and the inter-atrial septum. Mitral regurgitation refers to a condition in which the leaflets of the mitral valve do not seal properly. As a result, when the heart flexes in systole, the mitral valve does not close properly, allowing blood to regurgitate from the left ventricle into the left atrium. To compensate for this condition, the device 410 comprises a balloon 419 that is disposed at an intermediate position of a catheter tube 420. The structure of the device 410 is similar to the previously described embodiments in that the tubing 420 is hollow to allow a biocompatible fluid to flow between a pump 12 and the balloon 419. The balloon 419 may be inflated and deflated in synchronization with the systole and diastole rhythms as described before. That is, the balloon 419 is inflated during systole to assist in sealing the mitral valve as shown in FIG. 21. Similarly, the balloon 419 may be at least partially deflated in diastole to reduce the size of the balloon 419 and permit blood flow around the balloon 419 as shown in FIG. 20.

FIGS. 20 and 21 specifically show that the balloon 419 is disposed intermediate a left ventricular portion 422 of the tubing 320 and a left atrial portion 423 of the tubing 420. Anchor balloons 428, 430 are distal the left ventricle portion 422 of the tubing 420 and anchor the tubing 420 to the inter-ventricular septum. Similar anchors may be used at the inter-atrial septum, though none are specifically shown.

The device 410 may be installed in a manner similar to previously described approaches. One difference with this implementation is that an aperture is formed in the inter-atrial septum communicating both atria. In one implantation technique, devices similar to catheter 50, anchor member 52, and cutting member 54 may be used to form the inter-atrial aperture. Then, a second, slightly smaller catheter 50, anchor member 52, and cutting member 54 may be fed through the first aperture, through the mitral valve and to the left ventricle wall of the inter-ventricular septum to form a second aperture in the inter-ventricular septum. It should be noted, that the variations disclosed above for devices 110, 210, 310 used to treat the aortic regurgitation may be implemented with the device 410. For example, the balloon 419 may be filled and retained at a desired volume and pressure or may be inflated and deflated in synchronization with the systole and diastole rhythms of the heart. Further, in one embodiment, the balloon 419 and anchor balloons 428, 430 are in fluid communication with one another. In another embodiment, the balloon 419 may be inflated via a dedicated lumen separate from that used to fill the anchor balloons 428, 430.

Another aspect of the device 410 relates to the positioning of the balloon 419 relative to the Mitral Valve. Within the Left Ventrical, the Chordae Tendinae are attached to the leaflets of the Mitral Valve. Consequently, the balloon 419 may be positioned between the Chordinae Tendinae. In fact, the balloon 419 may be positioned so that it lies within the Mitral Valve during systole to effectively seal the Mitral Valve. Then, during diastole, the balloon 419 may move within the Left Ventricle to permit blood flow into the Left Ventricle.

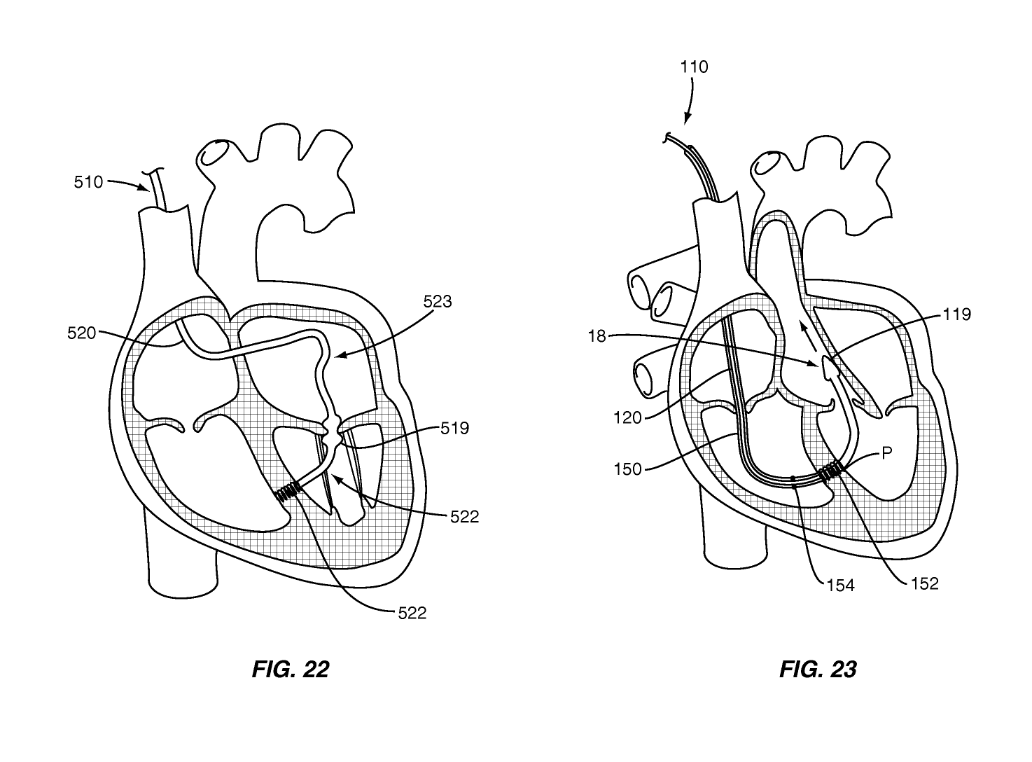

In embodiments described above, a catheter device may be secured to an inter-ventricular or inter-atrial septum via a pair of opposing anchor balloons. FIGS. 22 and 23 illustrate alternative approaches to anchoring the device. FIG. 22 depicts a device 510 similar to device 410 shown in FIGS. 20 and 21. Instead of anchor balloons 428, 430, the device 510 includes a coiled anchor member 552 disposed at its distal end. This coiled anchor member 552 may be similar to the coiled member 52 shown in FIGS. 8-11.

A similar anchor member 152 may be used in the device shown in FIG. 23. This device treats atrial regurgitation and includes two separate catheter lumens. An inner device 110 is similar to the device shown in FIGS. 12 and 13. This device 110 is contained within an outer tubing 150 that is similar to the insertion catheter 50 shown in FIGS. 8-11. The outer tubing 150 may be secured to the inter-atrial septum with a coiled anchor member 152 as described above. The inner device 110 may be secured to the patients anatomy or to the outer tubing 150, such as at connection point 154. The inner and outer tubes 120, 150 may be secured to one another with an adhesive, through interlocking features, or through inflation of an intermediate balloon (not specifically shown). Those skilled in the art will appreciate numerous alternative configurations for securing the inner and outer tubing 120, 150.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. For instance, certain embodiments used to assist systolic function and valve efficiency have been disclosed. The device may be used to assist in diastolic function by incorporating balloons in the left and/or right atria that inflate and deflate to improve flow during diastole. The present embodiments are, therefore, to be considered in all respects as illustrative and not restrictive, and all changes coming within the meaning and equivalency range of the appended claims are intended to be embraced therein.

What is claimed is:

1. An implant for insertion into a heart in a patient comprising:
an anchor portion including inflatable first and second anchor balloons, the first anchor balloon configured to be disposed on a first side of a cardiac septum and the second anchor balloon configured to be disposed on a second side of the cardiac septum, the first and second anchor balloons coupled to one another through an aperture in the cardiac septum;
an inflatable assist balloon to assist in cardiac function; and
catheter tubing including at least one lumen through which a fluid may be introduced to inflate the anchor balloons.

2. The implant of claim 1 wherein the assist balloon is an anchor balloon.

3. The implant of claim 1 wherein the assist balloon is in fluid communication with a first lumen in the catheter and the anchor balloons are in fluid communication with a second lumen in the catheter.

4. The implant of claim 1 further comprising:
a fluid reservoir to contain the fluid;
a pump to move the fluid between the fluid reservoir and the lumen;
a sensor to sense cardiac systole and diastole rhythms;
a controller to control the operation of the pump to move the fluid in synchronization with the cardiac systole and diastole rhythms.

5. The implant of claim 4 wherein the fluid reservoir, pump, sensor, and controller are configured for subcutaneous implantation.

6. The implant of claim 1 wherein the first anchor balloon is in fluid communication with a first lumen in the catheter and the second anchor balloon is in fluid communication with a second lumen in the catheter.

7. An implant for insertion into a heart in a patient comprising:
an anchor portion to be secured to the heart at a cardiac septum;
an inflatable assist balloon to assist in cardiac function, the assist balloon configured to assume a first shape during cardiac systole and to assume a second shape during cardiac diastole; and
catheter tubing including at least one lumen through which a fluid may be introduced to inflate the assist balloon.

8. The implant of claim 7 wherein the anchor portion includes a sharpened member to penetrate the septum.

9. The implant of claim 7 wherein the anchor portion includes a coiled member to penetrate the septum.

10. The implant of claim 7 wherein the anchor portion includes inflatable first and second anchor balloons configured to be coupled to one another through an aperture in the cardiac septum, the anchor balloons having a deflated width that is less than a width of the aperture and an inflated width that is greater than the width of the aperture.

11. The implant of claim 7 further comprising:
a fluid reservoir to contain the fluid;
a pump to move the fluid between the fluid reservoir and the lumen;
a sensor to sense cardiac systole and diastole rhythms;
a controller to control the operation of the pump to move the fluid in synchronization with the cardiac systole and diastole rhythms.

12. The implant of claim 11 wherein the controller controls the pump to inflate and deflate the assist balloon between the first and second shapes.

13. The implant of claim 12 wherein the controller controls the pump to inflate and deflate the assist balloon while the assist balloon is disposed in a ventricle, the assist balloon pumped to an inflated first shape during cardiac systole to increase systolic flow out of the ventricle, and the assist balloon pumped to a deflated second shape during cardiac diastole to permit filling of the ventricle.

14. The implant of claim 7 wherein the assist balloon is disposable adjacent a cardiac valve, the assist balloon assuming the first shape to seal the cardiac valve when the valve is closed, the assist balloon assuming the second shape to permit blood flow around the assist balloon when the cardiac valve is open.

15. The implant of claim 14 wherein the cardiac valve is an aortic valve and the assist balloon is disposed in the aorta.

16. The implant of claim 14 wherein the cardiac valve is a mitral valve and the assist balloon is disposed in the left atrium.

17. A method for assisting cardiac function in a patient comprising:
anchoring a catheter tubing to a cardiac septum; and
inflating an assist balloon to a predetermined pressure and volume to assist in cardiac function by causing the assist balloon to assume a first shape during cardiac systole and to assume a second shape during cardiac diastole.

18. The method of claim 17 wherein the step of anchoring the catheter to the cardiac septum comprises inflating a pair of opposed anchor balloons that are coupled to the tubing to a width that is greater than an aperture in the cardiac septum through which the anchor balloons are coupled.

19. The method of claim 17 wherein the step of anchoring the catheter to the cardiac septum comprises rotating a coiled anchor member that is coupled to the tubing into engagement with the cardiac septum.

20. The method of claim 17 further comprising positioning the assist balloon in a left ventricle, the assist balloon inflating during systole and deflating during diastole.

21. The method of claim 17 further comprising positioning the assist balloon in a left ventricle adjacent a cardiac mitral valve, the assist balloon assuming a first shape to seal the mitral valve during systole and assuming an elongated second shape during diastole to permit blood flow around the assist balloon.

22. The method of claim 21 further comprising inflating the assist balloon to the first shape and deflating the assist balloon to the second shape.

23. The method of claim 17 further comprising positioning the assist balloon in an aorta adjacent a cardiac aortic valve, the assist balloon assuming a first shape to seal the aortic valve during diastole and assuming an elongated second shape during systole to permit blood flow around the assist balloon.

24. The method of claim 23 further comprising inflating the assist balloon to the first shape and deflating the assist balloon to the second shape.

25. A method of implanting a cardiac assist device onto a cardiac septum within a heart of a patient comprising:
inserting a hollow first catheter into the heart and guiding the first catheter to the cardiac septum;
temporarily securing a distal end of the first catheter to the cardiac septum;
guiding a cutting tool through the first catheter and to the cardiac septum;
forming an aperture in the cardiac septum with the cutting tool;
removing the cutting tool from the first catheter;
guiding a second catheter including a balloon through the first catheter, the balloon being at least partially deflated;
guiding the balloon through the aperture and towards an opposite side of the cardiac septum, and
inflating the balloon to a balloon width that is greater than a width of the aperture.

26. The method of claim 25 wherein the step of guiding the second catheter including the balloon through the first catheter further comprises guiding the second catheter including a pair of balloons through the first catheter and positioning the pair of balloons through the aperture and on opposite sides of the cardiac septum.

27. The method of claim 25 further comprising removing the first catheter.

28. The method of claim 25 further comprising securing the first catheter to the second catheter.

29. The method of claim 25 wherein the cardiac septum is an inter-ventricular septum.

30. The method of claim 25 wherein the cardiac septum is an atrial septum.
